SEQUENCE LISTING (6 sequences)

SEQ ID NO: 1 - protein, 10 aa, synthetic construct
GYTFTSCVMN

SEQ ID NO: 2 - protein, 17 aa, synthetic construct
YINPYNDGTKYNEKFKG

SEQ ID NO: 3 - protein, 10 aa, synthetic construct
EGFYYGNFDN

SEQ ID NO: 4 - protein, 11 aa, synthetic construct
RASENIYSYLT

SEQ ID NO: 5 - protein, 7 aa, synthetic construct
NAKTLPE

SEQ ID NO: 6 - protein, 9 aa, synthetic construct
QHHYGTPYT

(19) United States
(12) Patent Application Publication
(10) Pub. No.: US 2026/0327340 A1
(43) Pub. Date: Oct. 22, 2026

(54) Methods to Regulate Activation of Transforming Growth Factor-beta via Targeting Alpha-Enolase for Treating Diseases

(71) Applicant: HUNILIFE BIOTECHNOLOGY, INC., Taipei City (TW)

(72) Inventors: TA-TUNG YUAN, Taipei City (TW); WEI-CHING HUANG, Taipei City (TW)

(21) Appl. No.: 19/653,250

(22) Filed: Apr. 21, 2026

Related U.S. Application Data
(60) Provisional application No. 63/791,888, filed on Apr. 21, 2025.

(51) Int. Cl. C07K 16/40; A61K 39/00; A61K 39/395; A61K 45/06; A61P 11/00; A61P 35/00

(52) U.S. Cl. CPC C07K 16/40; A61K 39/3955; A61K 45/06; A61P 11/00; A61P 35/00; C12Y 402/01011; A61K 39/0015; C07K 2317/24; C07K 2317/565

(57) ABSTRACT

Provided herein are methods for reducing the activation of TGF-β in tissues, comprising contacting the tissues of a subject with an alpha-enolase (ENO-1) monoclonal antibody.

### CROSS-REFERENCE TO RELATED APPLICATION

This application claims the benefit of U.S. Provisional Application No. 63/791,888 filed on Apr. 21, 2025, which is hereby incorporated by reference herein for all purposes.

### SEQUENCE LISTING STATEMENT

The contents of the electronic sequence listing titled 26P0233-seq-list.xml (Size: 6,537 bytes; and Date of Creation: Apr. 20, 2026) is herein incorporated by reference in its entirety.

### FIELD OF INVENTION

The disclosure relates to new methods for inhibiting the activation of Transforming Growth Factor-beta (TGF-β) by using an alpha-enolase (ENO-1) antagonist. More specifically, the disclosure relates to methods for controlling TGF-β activation by targeting extracellular ENO-1 or membrane-associated ENO-1. This approach aims to mitigate the toxicity issues associated with direct TGF-β inhibition, offering a novel therapeutic strategy for fibrosis, cancer, and other TGF-β-driven diseases.

### BACKGROUND OF THE INVENTION

Transforming Growth Factor-beta (TGF-β) is a multifunctional cytokine involved in immune regulation, fibrosis, and cancer progression. While TGF-β inhibition has been extensively explored as a therapeutic strategy, direct targeting of TGF-β or its receptors has encountered significant clinical challenges, including immune suppression, paradoxical tumor progression, and systemic toxicity. Monoclonal antibodies (e.g., fresolimumab) and small-molecule kinase inhibitors (e.g., galunisertib) designed to block TGF-β signaling have shown promise in preclinical models but have been limited by adverse effects, including impaired wound healing, increased susceptibility to infections, and dysregulated immune homeostasis. Additionally, in certain cancers, complete blockade of TGF-β can promote tumor progression by eliminating its tumor-suppressive functions in early-stage disease.

Enolase-1 (ENO-1) is a glycolytic enzyme with additional non-glycolytic functions that contribute to cellular signaling, immune modulation, and extracellular matrix remodeling. ENO-1 is upregulated in various pathological conditions, including fibrotic disorders and cancers, where it serves as a plasminogen receptor. In this role, ENO-1 facilitates the conversion of plasminogen to plasmin, enhancing plasmin-mediated fibrinolysis and promoting extracellular matrix (ECM) degradation, cell migration, and tissue remodeling.

### SUMMARY OF THE INVENTION

In the prior art, directly inhibiting TGF-β or its receptors frequently leads to severe systemic toxicity and immunosuppression by indiscriminately blocking basal TGF-β signaling throughout the body. To solve this objective technical problem, the present disclosure provides a novel, indirect therapeutic approach by selectively targeting extracellular or cell membrane-associated alpha-enolase (ENO-1). Unlike conventional direct inhibitors, targeting ENO-1 serves as a previously unrecognized upstream regulatory mechanism to modulate pathological TGF-β activation without shutting down its entire signaling cascade.

Specifically, the present disclosure disrupts TGF-β activation through a unique dual-pathway mechanism. First, it blocks ENO-1's function as a plasminogen receptor, thereby preventing the plasmin-mediated proteolytic cleavage of the latent TGF-β complex (LAP-TGF-β). Second, it inhibits ENO-1-driven glycolysis and subsequent lactate production, preventing the microenvironmental acidification that typically enhances integrin-mediated (e.g., αvβ6 and αvβ8) mechanical release of active TGF-β. By targeting ENO-1, both plasmin-mediated proteolysis and lactate-driven microenvironmental modulation are effectively disrupted at a critical extracellular checkpoint.

The technical effect achieved by this upstream strategy is highly advantageous and unexpected. Because the aberrant translocation of ENO-1 to the cell surface occurs predominantly in disease microenvironments (such as fibrotic tissues or tumor sites), the disclosed ENO-1 antagonists (e.g., monoclonal antibodies) selectively intercept pathological TGF-β activation while preserving physiological TGF-βfunctions in normal tissues. This distinct mechanism provides robust therapeutic efficacy against TGF-β-driven diseases-including fibrosis and cancer-with an unexpected and significantly superior safety profile compared to conventional direct TGF-β blockers.

The present invention relates to an alpha-enolase (enolase-1, ENO-1) antagonist targeting ENO-1 and a use thereof. In particular, the ENO-1 antagonist has binding affinity to ENO-1, such as human ENO-1, and may comprise an antigen-binding domain capable of neutralizing one or more biological activities of ENO-1. The ENO-1 antagonist can bind to soluble ENO-1 protein and/or ENO-1 expressed on the cell surface and is useful for modulating TGF-βactivation. Accordingly, the ENO-1 antagonist has significant potential in the treatment of TGF-β-driven diseases.

In accordance with certain embodiments of the disclosure, provided herein are methods for reducing activation of transforming growth factor-beta (TGF-β) in tissues, comprising contacting tissues of a subject with an ENO-1 antagonist. In some embodiments, the ENO-1 antagonist is a monoclonal antibody. In certain embodiments, the ENO-1 antagonist specifically binds to extracellular or membrane-associated ENO-1, thereby modulating TGF-βactivation in the extracellular matrix (ECM) and/or at the cell surface.

In accordance with certain embodiments of the disclosure, the ENO-1 antagonist inhibits TGF-β activation through one or more mechanisms, including: (a) blocking plasminogen binding to ENO-1, thereby reducing plasmin-mediated cleavage of latent TGF-β complexes; and/or (b) reducing lactate-induced extracellular acidification, thereby disrupting integrin-mediated TGF-β activation.

In accordance with certain embodiments of the disclosure, the methods described herein are applicable to tissues including, but not limited to, tumor tissues and lung tissues.

In accordance with certain embodiments of the disclosure, provided herein are methods for treating TGF-β-driven diseases, comprising administering to a subject in need thereof a therapeutically effective amount of an ENO-1 antagonist. In certain embodiments, the ENO-1 antagonist is a monoclonal antibody that binds to ENO-1 and inhibits plasminogen binding.

In accordance with certain embodiments of the disclosure, the ENO-1 antagonist may be administered via any suitable route, including but not limited to intraperitoneal, intravenous, subcutaneous, intramuscular, or other parenteral routes.

In accordance with certain embodiments of the disclosure, the therapeutically effective amount of the ENO-1 antagonist may range from about 5 to about 5000 mg/m², depending on factors such as disease type, subject condition, and route of administration.

In accordance with certain embodiments of the disclosure, the ENO-1 antagonist comprises a monoclonal antibody including a heavy chain variable domain (VH) and a light chain variable domain (VL), wherein the VH comprises complementarity-determining regions (CDRs) including HCDR1 (GYTFTSCVMN; SEQ ID NO: 1), HCDR2 (YINPYNDGTKYNEKFKG; SEQ ID NO: 2), and HCDR3 (EGFYYGNFDN; SEQ ID NO: 3), and the VL comprises complementarity-determining regions including LCDR1 (RASENIYSYLT; SEQ ID NO: 4), LCDR2 (NAKTLPE; SEQ ID NO: 5), and LCDR3 (QHHYGTPYT; SEQ ID NO: 6), as defined by SEQ ID NOs: 1-6.

In accordance with certain embodiments of the disclosure, the ENO-1 antagonist may be used in combination therapy with one or more additional therapeutic agents, including anti-fibrotic agents, immune modulators, or TGF-β pathway inhibitors.

In accordance with certain embodiments of the disclosure, the TGF-β-driven diseases include, but are not limited to, multiple myeloma, pancreatic cancer, and idiopathic pulmonary fibrosis.

Compared with conventional approaches, the present invention provides a therapeutic strategy targeting ENO-1 that simultaneously modulates plasmin-associated proteolysis, metabolic reprogramming, and TGF-β activation. This coordinated multi-pathway regulation provides a mechanistic advantage and supports the use of ENO-1 antagonists in treating diseases associated with aberrant TGF-βsignaling.

One skilled in the art would appreciate that the therapeutically effective amount and administration regimen may vary depending on factors such as the subject's condition, age, disease severity, and route of administration, and may be determined without undue experimentation.

Other aspects of the present invention will become apparent from the following detailed description.

### BRIEF DESCRIPTION OF THE DRAWINGS

FIG. 1, part A, shows plasmin activity following treatment with an ENO-1 monoclonal antibody.

FIG. 1, part B, shows extracellular lactate levels following treatment with an ENO-1 monoclonal antibody.

FIG. 1, part C, shows levels of active TGF-βfollowing treatment with an ENO-1 monoclonal antibody.

FIG. 1, part D, shows expression of fibroblast activation protein (FAP) following inhibition of the TGF-β pathway.

FIG. 2, part A, shows tumor weight following treatment with an ENO-1 monoclonal antibody in an orthotopic PDAC model.

FIG. 2, part B, shows tumor lactate levels following treatment with an ENO-1 monoclonal antibody.

FIG. 2, part C, shows levels of active TGF-β in tumor tissues following treatment with an ENO-1 monoclonal antibody.

FIG. 3, part A, shows plasmin activity in PANC-1/M cells upon TGF-β stimulation and ENO-1 monoclonal antibody treatment.

FIG. 3, part B, shows levels of active TGF-β in a co-culture system of PANC-1/M cells and THP-1 cells following ENO-1 monoclonal antibody treatment.

FIG. 4, part A, shows activation of SMAD3 in lung tissues following ENO-1 monoclonal antibody treatment in a pulmonary fibrosis model.

FIG. 4, part B, shows α-smooth muscle actin (α-SMA) expression in lung tissues following ENO-1 monoclonal antibody treatment in a pulmonary fibrosis model.

FIG. 4, part C, shows collagen deposition in lung tissues following ENO-1 monoclonal antibody treatment in a pulmonary fibrosis model.

FIG. 5, part A, shows tumor weight in an MBT-2 syngeneic mouse bladder cancer model following ENO-1 monoclonal antibody treatment.

FIG. 5, part B, shows serum interleukin-15 (IL-15) levels following ENO-1 monoclonal antibody treatment.

FIG. 5, part C, shows intratumoral Granzyme B (Gzmb) expression following ENO-1 monoclonal antibody treatment.

### DETAILED DESCRIPTION

The practice of the present invention will employ, unless otherwise indicated, conventional techniques of molecular biology, microbiology, recombinant DNA technology, protein chemistry, cell biology, and immunology, all of which are within the skill of the art. Such techniques include, but are not limited to, nucleic acid amplification, cloning, expression of recombinant proteins, cell culture, purification and characterization of proteins, antigen-antibody interaction assays, and immunodetection methods. These methodologies are well known in the art and are described in various standard references and therefore need not be described in detail herein. It will be understood that variations and modifications of these techniques may be employed by those skilled in the art without departing from the scope of the present invention.

The term “ENO-1 antagonist” as used herein, refers to any molecule or substance that is capable of binding to ENO-1 (enolase-1) as an antigen or target molecule and, upon such binding, inhibits, blocks, reduces, or otherwise neutralizes one or more biological activities or functions of ENO-1. The antagonist may exert its effect by directly interfering with the functional domain of ENO-1, preventing its interaction with other binding partners, or altering its structural conformation. Examples of ENO-1 antagonists include antibodies or other binding agents capable of specific interaction with ENO-1. The degree of inhibition may vary and includes partial or complete suppression of ENO-1 activity.

The terms “antibody” and “immunoglobulin” are used herein interchangeably in their broadest sense and encompass any immunoglobulin molecule capable of specifically binding to an antigen, including ENO-1. Such antibodies include monoclonal antibodies (e.g., full-length or intact monoclonal antibodies), provided that they exhibit the desired biological activity. Antibodies described herein may be of any species origin, including murine, chimeric, humanized, or fully human antibodies, and may be generated or modified by techniques such as affinity maturation or other recombinant methods to enhance binding affinity, specificity, or functional activity.

The term “variable domain” refers to the sequence heterogeneity observed within specific segments of an antibody, which dictates its antigenic specificity and binding affinity. This variability is non-uniformly distributed, being primarily localized within three hypervariable segments known as complementarity-determining regions (CDRs). Conversely, the more conserved sequences, designated as framework regions (FR), consist of four segments that predominantly adopt a beta-sheet conformation. These FRs act as a structural scaffold, positioning the three CDR loops in spatial proximity. Together, the CDRs from both heavy and light chains converge to form the antigen-binding site. While the constant domains do not participate in antigen recognition, they mediate various effector functions, including antibody-dependent cellular cytotoxicity (ADCC).

As used herein, “treatment” or “treating” refers to any intervention intended to alter the course of a disease, disorder, or condition, including prophylactic and therapeutic applications. Treatment may include preventing, delaying, alleviating, or reducing one or more symptoms or effects of a disease, inhibiting disease progression, or otherwise improving a subject's condition.

An “individual” or a “subject” is a vertebrate. In certain embodiments, the vertebrate is a mammal. Mammals include, but are not limited to, farm animals (such as cows), sport animals, pets (such as cats, dogs, horses), primates, mice, and rats. In certain embodiments, the vertebrate is a human.

A “therapeutically effective amount” refers to an amount of a substance or molecule sufficient, at appropriate dosages and for a suitable period of time, to achieve a desired therapeutic or prophylactic effect. The effective amount may vary depending on factors including the disease condition, age, sex, weight, and overall health of the individual, as well as the properties of the substance or molecule and its ability to elicit a desired response.

In some embodiments, the therapeutically effective amount is one in which any adverse effects are outweighed by the therapeutic benefits. In one embodiment, the effective amount of the anti-ENO-1 antibody ranges from about 5 to about 5000 mg/m², such as about 50 to about 2500 mg/m², or about 100 to about 1000 mg/m², including, for example, about 5, 50, 100, 1000, 2000, 3000, 4000, or 5000 mg/kg.

The term “TGF-β-driven diseases” as used herein refers to diseases or disorders in which transforming growth factor-beta (TGF-β) signaling plays a role in the initiation, progression, or maintenance of the disease. Such diseases include, but are not limited to, cancer and tumor progression, or fibrotic disorders (e.g., pulmonary, hepatic, renal, or cardiac fibrosis). In such diseases, dysregulation or overactivation of the TGF-β pathway may contribute to pathological processes including excessive extracellular matrix deposition, immune modulation, and tissue remodeling. Accordingly, modulation or inhibition of TGF-β signaling may provide therapeutic or prophylactic benefit. In certain embodiments, the TGF-β-driven diseases include, but are not limited to, cancer and fibrotic diseases. In some embodiments, the diseases include multiple myeloma, pancreatic cancer, pulmonary fibrosis. (IPF), and bladder tumor.

In an embodiment, the ENO-1 antagonist may be administered with anti-fibrotic agents, immune modulators, or TGF-β pathway inhibitors for the treatment of TGF-β-driven diseases. The medicaments of the present disclosure may be applied locally or systemically. The medicaments of the present disclosure may also be supplied in combinations or with cofactors. ENO-1 antagonist of the present disclosure may be administered in an amount sufficient to restore normal levels, if the ENO-1 antagonist of the present disclosure is normally present in the target location, or they may be administered in an amount to raise levels above normal levels in the target location.

The medicaments of the present disclosure may be supplied to a target location from an exogenous source, or they may be made in vivo by cells in the target location or cells in the organism as the target location.

ENO-1 antagonist of the present disclosure may be in any physiologically appropriate formulation. They may be administered to a subject by intraperitoneal injection, or by any other effective means.

### Methods for Reducing the Activation of TGF-β or Inhibiting TGF-β-Driven Diseases.

The present application is based, at least in part, on the finding that an ENO-1 antagonist can specifically bind to extracellular or membrane-associated ENO-1. In certain embodiments, such binding modulates TGF-β activity in the extracellular matrix (ECM) or on the cell surface.

In one embodiment, a method for reducing the activation of TGF-β in a tissue is provided. The method comprises contacting a tissue of a subject (e.g., tumor tissue or lung tissue) with an ENO-1 antagonist, thereby allowing the ENO-1 antagonist to bind to ENO-1. In certain embodiments, such binding interferes with the interaction between plasminogen and ENO-1, thereby reducing plasmin-mediated cleavage of a latent TGF-β complex. In some embodiments, the ENO-1 antagonist further reduces lactate-induced extracellular acidification, thereby decreasing integrin-mediated activation of TGF-β.

In one embodiment, a method for inhibiting TGF-β-driven diseases is provided, comprising administering to a subject in need thereof a therapeutically effective amount of an ENO-1 antagonist, thereby reducing TGF-β activation.

Embodiments of the present disclosure will be illustrated with the following specific examples. One skilled in the art would appreciate that these examples are for illustration only and that other modifications and variations are possible without departing from the scope of the present disclosure.

### EXAMPLES

### Example 1. Preparation of Anti-ENO-1 Antibody

In accordance with embodiments of the present disclosure, a general method for producing anti-ENO-1 antibodies comprises obtaining a hybridoma capable of producing a monoclonal antibody specific to ENO-1. Methods for generating monoclonal antibodies are well established in the art and are not described in detail herein. Briefly, mice may be immunized with ENO-1 antigen in the presence of a suitable adjuvant. Thereafter, spleen cells from the immunized mice are collected and fused with myeloma cells to generate hybridomas. Positive hybridoma clones may be identified based on their ability to bind the ENO-1 antigen using any suitable assay, such as enzyme-linked immunosorbent assay (ELISA).

In one embodiment, the anti-ENO-1 antibody comprises a heavy chain variable domain having three complementarity-determining regions (CDRs), including HCDR1 (GYTFTSCVMN; SEQ ID NO: 1), HCDR2 (YINPYNDGTKYNEKFKG; SEQ ID NO: 2), and HCDR3 (EGFYYGNFDN; SEQ ID NO: 3), and a light chain variable domain having three complementarity-determining regions, including LCDR1 (RASENIYSYLT; SEQ ID NO: 4), LCDR2 (NAKTLPE; SEQ ID NO: 5), and LCDR3 (QHHYGTPYT; SEQ ID NO: 6).

In accordance with embodiments of the present disclosure, the antibodies may be mouse antibodies. Alternatively, the antibodies may be chimeric antibodies, such as antibodies comprising human constant regions linked to mouse variable regions, humanized antibodies in which mouse CDRs are grafted onto human immunoglobulin framework regions, or fully human antibodies.

In one embodiment, the monoclonal antibody may be humanized by obtaining the CDR sequences from the hybridoma and grafting the CDR sequences onto human framework sequences to generate humanized antibodies. Any suitable methods known in the art for identifying CDR sequences may be used. In certain embodiments, the CDR regions of the present invention are identified according to the Kabat numbering scheme. A hybridoma producing anti-ENO-1 antibody (e.g., a mouse hybridoma) is first generated using standard monoclonal antibody production techniques. Total RNA is then isolated from the hybridoma, for example using TRIzol® reagent. Complementary DNA (cDNA) is subsequently synthesized from the total RNA, for example using a first-strand cDNA synthesis kit (SuperScript™ III) with an oligo (dT20) primer or an Ig-3′ constant region primer.

The variable regions of the immunoglobulin heavy and light chains are then cloned from the complementary DNA (cDNA). For example, VH and VL regions of the anti-ENO-1 monoclonal antibody may be amplified from mouse hybridoma cDNA by polymerase chain reaction (PCR) using a mouse Ig-5′ primer set (Novagen). The resulting PCR products may be directly cloned into a suitable vector, such as a pJET1.2 vector, using a CloneJET™ PCR Cloning Kit (Fermentas). The pJET1.2 vector contains a lethal gene cassette such that only recombinant vectors containing the inserted target sequence can be propagated under selection, thereby facilitating selection of positive clones. Recombinant colonies are then screened to identify desired clones, followed by plasmid isolation and sequencing. The immunoglobulin nucleotide sequences may be analyzed using the International ImMunoGeneTics (IMGT) information system.

### Antibody Expression and Purification.

For antibody production, the isolated expression clones may be introduced into suitable host cells for expression. In one example, F293 cells (Life Technologies) are transfected with a plasmid encoding the anti-ENO-1 monoclonal antibody and cultured for approximately 7 days. The expressed anti-ENO-1 antibody is then purified from the culture supernatant using a protein A affinity column (GE Healthcare). Protein concentration may be determined using a Bio-Rad protein assay kit, and analyzed by 12% SDS-PAGE, according to standard procedures or the manufacturer's instructions.

### Example 2. In Vitro Suppression of Plasmin Activity, Lactate Production, and TGF-β Activation by ENO-1 mAb in MM-Conditioned Stromal Cells

In this embodiment, HS-5 human bone marrow stromal cells were cultured under conditions designed to recapitulate a tumor-associated stromal microenvironment, including supplementation with multiple myeloma (MM) cell-conditioned supernatant and recombinant ENO-1 protein. The cells were subsequently contacted with an ENO-1 mAb at a concentration of 100 μg/mL or a control IgG for 24 or 48 hours.

To investigate the effects of ENO-1 antagonism on key mediators of TGF-β activation and matrix activation in human bone marrow stromal cells (HS-5), plasmin activity, extracellular lactate production, Active TGF-β levels, and fibroblast activation protein (FAP) expression were measured. In this embodiment, plasmin activity was measured by chromogenic substrate assay. Lactate production in the culture supernatant was measured by lactate colorimetric assay. Active TGF-β levels were quantified by ELISA. In a parallel experimental setup, cells were treated with a TGF-β receptor inhibitor, and fibroblast activation protein (FAP) expression was analyzed by Western blotting.

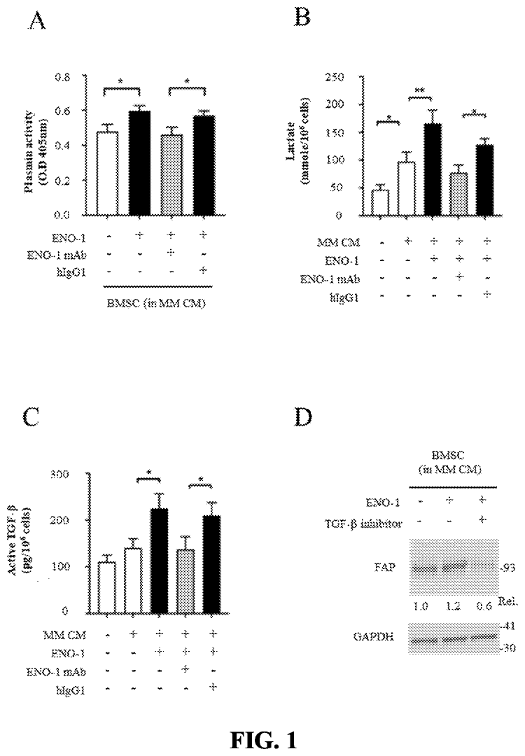

As shown in FIG. 1, part A, plasmin activity in HS-5 stromal cells cultured under MM-conditioned conditions was measured by chromogenic substrate assay. Treatment with ENO-1 mAb (100 μg/ml) resulted in a marked reduction in plasmin activity relative to control IgG-treated cells, indicating that ENO-1 function is required for maintaining plasmin-associated proteolytic activity in the tumor stromal microenvironment.

As shown in FIG. 1, part B, lactate concentration in the culture supernatant was measured by lactate colorimetric assay. ENO-1 mAb (100 μg/ml) treatment decreased extracellular lactate levels, demonstrating that inhibition of ENO-1 concurrently suppresses metabolic activity associated with glycolytic activity.

As shown in FIG. 1, part C, active TGF-β levels were measured by ELISA. A reduction in active TGF-β was observed after ENO-1 mAb (100 μg/ml) treatment, suggesting that ENO-1 activity contributes to the activation of latent TGF-β, likely through a plasmin-dependent proteolytic mechanism.

As shown in FIG. 1, part D, fibroblast activation protein (FAP) expression was measured by Western blotting with a TGF-β receptor inhibitor treatment. Inhibition of TGF-β signaling resulted in decreased FAP expression, confirming that TGF-β activity is functionally required for stromal cell activation.

Taken together, the results shown in FIGS. 1A to 1D support a mechanistic cascade in which ENO-1 functions upstream to regulate plasmin activity and metabolic output, thereby promoting activation of TGF-β and subsequent stromal activation. In particular, inhibition of ENO-1 simultaneously inhibits proteolytic activity, metabolic reprogramming, and TGF-β signaling, demonstrating a coordinated regulatory role that is not limited to a single pathway. This multi-level modulation indicates that ENO-1 serves as a key regulator of proteolysis, metabolism, and cytokine activation within the tumor microenvironment.

### Example 3. In Vivo Reduction of Tumor Burden and TGF-β Activation

in an Orthotopic Pancreatic Cancer Model Treated with ENO-1 mAb.

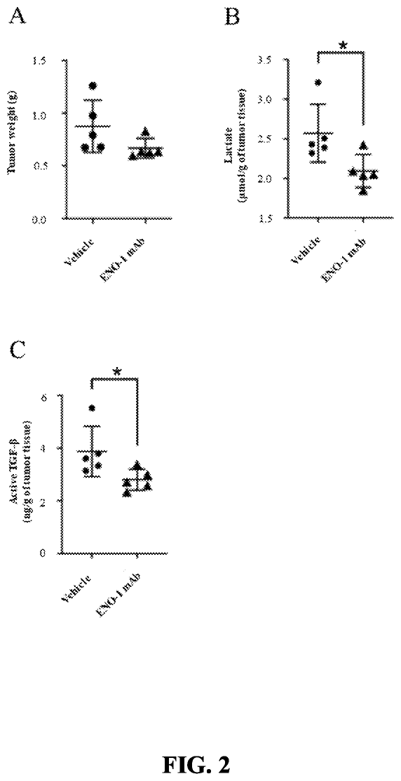

As shown in FIG. 2, part A, orthotopic pancreatic tumors were established in immunodeficient mice by surgical implantation of PANC-1/M cells into the pancreas. Upon tumor establishment, the animals were randomized to receive an anti-ENO-1 monoclonal antibody (ENO-1 mAb) at 30 mg/kg or a vehicle control, administered twice weekly via intraperitoneal injection. At the study endpoint, tumors were excised and weighed to assess tumor burden. ENO-1 mAb-treated animals exhibited a reduction in tumor weight relative to vehicle-treated controls, indicating that inhibition of ENO-1 suppresses tumor growth in vivo.

As shown in FIG. 2, part B, tumor tissues collected at the endpoint were analyzed for lactate content using a colorimetric assay. The lactate levels in the tumors from ENO-1 mAb-treated animals were decrease, demonstrating that ENO-1 inhibition attenuates tumor-associated glycolytic activity and metabolic rate.

As shown in FIG. 2, part C, active TGF-β levels in tumor tissues were quantified by ELISA. A reduction in active TGF-β levels was observed in ENO-1 mAb-treated animals, indicating that ENO-1 activity contributes to TGF-β activation within the tumor microenvironment.

Taken together, the results shown in FIGS. 2A to 2C show a mechanistic relationship in which ENO-1 functions upstream to cooperate with tumor growth, metabolic activity, and TGF-β activation. In particular, inhibition of ENO-1 concurrently reduces tumor size, lactate accumulation, and active TGF-β levels, demonstrating that ENO-1 regulates an integrated metabolic and cytokine signaling pathway in vivo.

### Example 4. Reciprocal Regulation of Plasmin and TGF-β Activity Via ENO-1 in Pancreatic Cancer Cells

To evaluate the reciprocal regulatory relationship between plasmin activity and TGF-β activation in the tumor microenvironment, the pancreatic cancer cell line PANC-1/M was used in conjunction with recombinant TGF-β stimulation or co-culture with THP-1 monocytic cells to mimic tumor-associated macrophage (TAM) influence. The role of ENO-1 in mediating this regulatory interaction was assessed using a neutralizing monoclonal antibody against ENO-1 (ENO-1 mAb), each at concentrations of 1, 10, and 100 μg/mL.

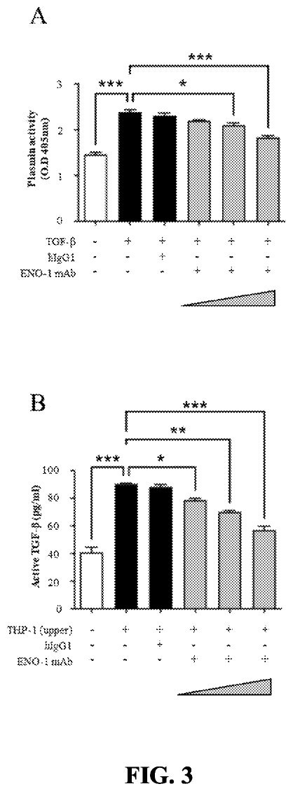

As shown in FIG. 3, part A, PANC-1/M cells were treated with exogenous TGF-β (50 ng/mL) in the presence or absence of control immunoglobulin (hIgG1) or ENO-1 mAb (1, 10, 100 μg/ml) at increasing concentrations for 48 hours. Plasmin activity was measured using a chromogenic substrate assay by measuring the absorbance at OD405 nm. TGF-β stimulation increased plasmin activity relative to untreated controls, whereas ENO-1 mAb reduced plasmin activity in a dose-dependent manner, indicating that ENO-1 activity is required for maintaining TGF-β-induced plasmin generation.

As shown in FIG. 3, part B, PANC-1/M cells were co-cultured with THP-1 cells using a Transwell assay, wherein THP-1 cells were seeded in the upper chamber and PANC-1/M cells were maintained in the lower chamber. The co-culture was performed in the presence or absence of control IgG or ENO-1 mAb (1, 10, 100 μg/ml) at increasing concentrations for 72 hours. Conditioned media from the lower chamber were collected and analyzed for active TGF-β levels by ELISA. Co-culture with THP-1 cells increased active TGF-β levels relative to monoculture conditions, whereas ENO-1 mAb treatment reduced active TGF-β levels in a dose-dependent manner, indicating that ENO-1 contributes to macrophage-associated enhancement of TGF-β activation.

As shown in FIG. 3, the combined results from FIGS. 3A and 3B demonstrate a reciprocal regulatory relationship between plasmin activity and TGF-β activation mediated by ENO-1. In particular, ENO-1 facilitates plasmin-associated proteolytic activity that contributes to activation of latent TGF-β, while TGF-β signaling enhances plasmin activity, thereby forming a feed-forward loop. Inhibition of ENO-1 disrupts both arms of this loop, decreasing plasmin activity and active TGF-β levels. These findings support a mechanistic model in which ENO-1 is involved in regulating plasmin activity, metabolic processes, and TGF-β activation coordinating proteolytic and cytokine signaling pathways within the tumor microenvironment.

### Example 5. ENO-1 mAb Ameliorates Pulmonary Fibrosis in Bleomycin-Induced Lung Injury Model

In this embodiment, C57BL/6 mice were administered bleomycin (1.8 U/kg, intratracheally) to induce pulmonary fibrosis. Starting on day 7 post-instillation, mice were treated with an ENO-1 mAb at a dose of 30 mg/kg or a vehicle control, administered twice per week via intraperitoneal injection. Lung tissues were harvested on day 21 and processed for preparation of frozen sections for histological analysis.

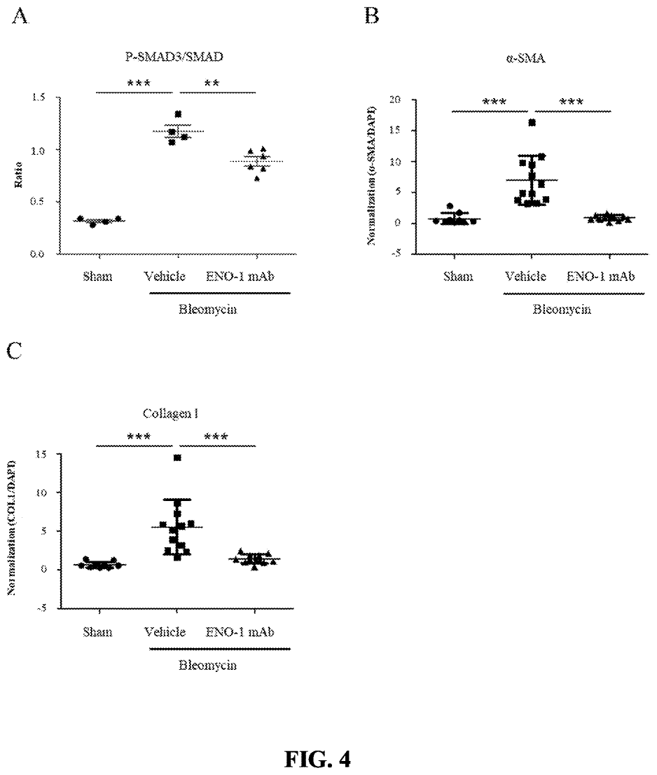

As shown in FIG. 4, part A, lung tissue extracts were subjected to Western blotting analysis to evaluate activation of TGF-β signaling by measuring phosphorylation of SMAD family member 3 (SMAD3), and ENO-1 mAb-treated mice exhibited reduced SMAD3 activation compared to vehicle-treated controls, indicating attenuation of TGF-β signaling.

As shown in FIG. 4, part B, immunofluorescence staining was performed on lung tissue sections to evaluate α-smooth muscle actin (α-SMA) expression as a marker of myofibroblast activation. ENO-1 mAb-treated mice exhibited reduced α-SMA-positive staining compared to vehicle-treated controls, indicating suppression of myofibroblast activation.

As shown in FIG. 4, part C, collagen deposition in lung tissue was assessed as an indicator of extracellular matrix remodeling. ENO-1 mAb-treated mice displayed decreased collagen accumulation relative to controls, demonstrating suppression of fibrotic matrix deposition.

Taken together, the results shown in FIGS. 4A to 4C indicate that ENO-1 mAb-treated mice exhibited reduced phosphorylation of SMAD3, a downstream effector of TGF-β signaling, indicating attenuation of TGF-β activation. Correspondingly, ENO-1 mAb-treated mice showed reduced myofibroblast accumulation, as evidenced by decreased α-SMA-positive staining, and decreased collagen deposition, indicating reduced extracellular matrix remodeling. These findings collectively indicate that inhibition of ENO-1 is associated with suppression of key fibrotic processes in a bleomycin-induced pulmonary fibrosis model and is consistent with modulation of TGF-β-driven fibrotic signaling pathways.

In prior studies, ENO-1 inhibition has been associated with reduced lactate accumulation and decreased active TGF-β levels in bronchoalveolar lavage fluid (BALF) from bleomycin-challenged mice. In the context of the present disclosure, the observed reductions in α-SMA expression and collagen deposition, together with previously observed decreases in TGF-β activation, support a mechanistic model in which ENO-1 functions upstream to regulate TGF-β-driven fibrotic responses.

Notably, the concurrent attenuation of myofibroblast activation and extracellular matrix deposition upon ENO-1 inhibition represents a coordinated anti-fibrotic effect that would not have been reasonably expected from targeting a molecule primarily characterized as a glycolytic enzyme. These findings indicate that ENO-1 plays a broader regulatory role in fibrosis-related signaling networks and support the non-obviousness of targeting ENO-1 for treatment of TGF-β-driven fibrotic diseases. In summary, the data presented in the foregoing Examples demonstrate that inhibition of ENO-1 using a neutralizing ENO-1 mAb produces consistent modulatory effects across multiple in vitro and in vivo systems, including tumor-associated stromal models, tumor xenograft models, and bleomycin-induced pulmonary fibrosis models.

### Example 6. In Vivo Anti-Tumor Efficacy and Immune Modulation of ENO-1 mAb in a MBT-2 Syngeneic Mouse Bladder Cancer Model

The in vivo anti-tumor activity of the ENO-1 mAb was evaluated using an MBT-2 syngeneic murine bladder tumor model. MBT-2 cells were cultured under standard conditions and subcutaneously implanted into the right flank of immunocompetent C3H mice. When tumors reached an average volume of approximately 50 to 100 mm³, the mice were randomized into three groups: a vehicle control group, a 3 mg/kg ENO-1 mAb treatment group, and a 10 mg/kg ENO-1 mAb treatment group. The ENO-1 mAb was administered via intraperitoneal injection twice weekly. Tumor growth and body weight were monitored throughout the study. At the study endpoint, the mice were sacrificed, and the tumors were excised and weighed. Blood samples were collected to obtain serum for cytokine analysis, and a portion of the tumor tissue was snap-frozen for subsequent quantitative PCR (qPCR) analysis.

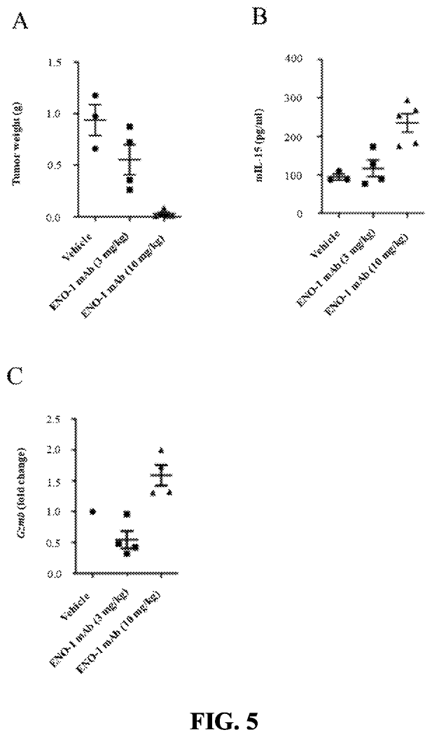

To further investigate mechanisms associated with the observed anti-tumor effects, pathways related to tumor-associated immune suppression were examined. Given that TGF-β signaling is associated with suppression of immunostimulatory cytokines, serum levels of mouse interleukin-15 (mIL-15) were measured. mIL-15 plays an important role in the survival and activation of natural killer (NK) cells and CD8⁺ T cells. As shown in FIG. 5B, ENO-1 mAb treatment was associated with a dose-dependent increase in serum mIL-15 levels compared to the vehicle control, suggesting a reduction in systemic immune suppression.

The local immune response within the tumor microenvironment was further evaluated by measuring the mRNA expression of Granzyme B (Gzmb), a cytotoxic effector molecule produced by T cells and NK cells. qPCR analysis demonstrated increased Gzmb expression in tumor tissues from ENO-1 mAb-treated mice, particularly at the 10 mg/kg dose (FIG. 5C). The observed increase in both systemic mIL-15 levels and intratumoral Gzmb expression indicates that ENO-1 inhibition is associated with enhancement of anti-tumor immune activity, consistent with modulation of TGF-β-mediated immunosuppressive signaling.

In particular, ENO-1 inhibition was found to reduce plasmin activity, decrease extracellular lactate production, and suppress activation of TGF-β, accompanied by reduced tumor burden in an orthotopic pancreatic cancer model and attenuation of fibrotic hallmarks such as myofibroblast activation and collagen deposition in a pulmonary fibrosis model, and suppression of tumor growth in an MBT-2 syngeneic murine bladder tumor model. In the MBT-2 model, ENO-1 mAb treatment was further associated with increased systemic levels of IL-15 and elevated intratumoral expression of Gzmb, indicating enhancement of anti-tumor immune responses. These coordinated effects across distinct pathological contexts support a mechanistic role of ENO-1 as an upstream regulator of proteolytic activity, metabolic reprogramming, and TGF-β activation, and indicate that targeting ENO-1 provides a unified therapeutic approach for diseases associated with aberrant TGF-β signaling.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

### EMBODIMENTS

[Embodiment 1] A method for reducing the activation of TGF-β in tissues, including: contacting the tissues of a subject with an alpha-enolase (ENO-1) monoclonal antibody.
[Embodiment 2] The method of any one of the precedent embodiments, wherein the ENO-1 monoclonal antibody specifically binds to extracellular or membrane-associated ENO-1.
[Embodiment 3] The method of any one of the precedent embodiments, wherein the ENO-1 monoclonal antibody inhibits TGF-β activation through at least one of the following mechanisms:
a) blocking plasminogen binding to ENO-1, reducing plasmin-mediated cleavage of the latent TGF-β complex; and/or
b) reducing lactate-induced extracellular acidification, disrupting integrin-mediated TGF-β activation.
   [Embodiment 4] The method of any one of the precedent embodiments, wherein the tissues are tumor tissues or lung tissues.
   [Embodiment 5] A method for treating TGF-β-driven diseases, including:
administering to a subject in need thereof a therapeutically effective amount of an ENO-1 monoclonal antibody.
   [Embodiment 6] The method of any one of the precedent embodiments, wherein the ENO-1 monoclonal antibody specifically binds to extracellular or membrane-associated ENO-1.
   [Embodiment 7] The method of any one of the precedent embodiments, further including inhibiting TGF-β activation by blocking plasminogen binding to ENO-1 and/or reducing lactate-induced extracellular acidification.
   [Embodiment 8] The method of any one of the precedent embodiments, wherein the therapeutically effective amount of the ENO-1 monoclonal antibody ranges from 5 to 5000 mg/m².
   [Embodiment 9] The method of any one of the precedent embodiments, wherein the ENO-1 monoclonal antibody is administered via intraperitoneal injection.
   [Embodiment 10] The method of any one of the precedent embodiments, wherein the ENO-1 monoclonal antibody binds to ENO-1 and inhibits plasminogen binding.
   [Embodiment 11] The method of any one of the precedent embodiments, wherein the ENO-1 monoclonal antibody comprises:
a heavy chain variable domain (VH) having three complementarity-determining regions including:
   a heavy chain complementarity determining region 1 (HCDR1) having the amino acid sequence of SEQ ID NO: 1;
   HCDR2 having the amino acid sequence of SEQ ID NO: 2; and
   HCDR3 having the amino acid sequence of SEQ ID NO: 3; and
a light chain variable domain (VL) having three complementarity-determining regions including
   a light chain complementarity determining region 1 (LCDR1) having the amino acid sequence of SEQ ID NO: 4;
   LCDR2 having the amino acid sequence of SEQ ID NO: 5; and
   LCDR3 having the amino acid sequence of SEQ ID NO: 6.
      [Embodiment 12] The method of any one of the precedent embodiments, wherein the ENO-1 monoclonal antibody is used in a combination therapy with one or more additional therapeutic agents, including: anti-fibrotic agents, immune modulators, or TGF-β pathway inhibitors.
      [Embodiment 13] The method of any one of the precedent embodiments, wherein the TGF-β-driven diseases comprise multiple myeloma, pancreatic cancer, idiopathic pulmonary fibrosis (IPF), bladder cancer, or urothelial carcinoma.
      [Embodiment 14] An ENO-1 monoclonal antibody for use in treating a TGF-β-driven disease in a subject.
      [Embodiment 15] The ENO-1 monoclonal antibody for use of any one of the precedent embodiments, wherein the TGF-β-driven diseases comprise multiple myeloma, pancreatic cancer, idiopathic pulmonary fibrosis (IPF), bladder cancer, or urothelial carcinoma.
      [Embodiment 16] The ENO-1 monoclonal antibody for use of any one of the precedent embodiments, wherein the ENO-1 monoclonal antibody comprises:
a heavy chain variable domain (VH) having three complementarity-determining regions including:
   a heavy chain complementarity determining region 1 (HCDR1) having the amino acid sequence of SEQ ID NO: 1;
   HCDR2 having the amino acid sequence of SEQ ID NO: 2; and
   HCDR3 having the amino acid sequence of SEQ ID NO: 3; and
a light chain variable domain (VL) having three complementarity-determining regions including
   a light chain complementarity determining region 1 (LCDR1) having the amino acid sequence of SEQ ID NO: 4;
   LCDR2 having the amino acid sequence of SEQ ID NO: 5; and
   LCDR3 having the amino acid sequence of SEQ ID NO: 6.
      [Embodiment 17] Use of an ENO-1 monoclonal antibody in the manufacture of a medicament for treating a TGF-β-driven disease in a subject.

What is claimed is:

1. A method for reducing the activation of transforming growth factor-beta (TGF-β) in tissues, comprising:
contacting the tissues of a subject with an alpha-enolase (ENO-1) monoclonal antibody.

2. The method of claim 1, wherein the ENO-1 monoclonal antibody specifically binds to extracellular or membrane-associated ENO-1.

3. The method of claim 1, wherein the ENO-1 monoclonal antibody inhibits TGF-β activation through at least one of the following mechanisms:
a) blocking plasminogen binding to ENO-1, reducing plasmin-mediated cleavage of the latent TGF-β complex; and/or
b) reducing lactate-induced extracellular acidification, disrupting integrin-mediated TGF-β activation.

4. The method of claim 1, wherein the tissues are tumor tissues or lung tissues.

5. A method for treating TGF-β-driven diseases, comprising:
administering to a subject in need thereof a therapeutically effective amount of an ENO-1 monoclonal antibody.

6. The method of claim 5, wherein the ENO-1 monoclonal antibody specifically binds to extracellular or membrane-associated ENO-1.

7. The method of claim 5, further comprising inhibiting TGF-β activation by blocking plasminogen binding to ENO-1 and/or reducing lactate-induced extracellular acidification.

8. The method of claim 5, wherein the therapeutically effective amount of the ENO-1 monoclonal antibody ranges from 5 to 5000 mg/m².

9. The method of claim 5, wherein the ENO-1 monoclonal antibody is administered via intraperitoneal injection.

10. The method of claim 5, wherein the ENO-1 monoclonal antibody binds to ENO-1 and inhibits plasminogen binding.

11. The method of claim 10, wherein the monoclonal antibody comprises:
a heavy chain variable domain (VH) having three complementarity-determining regions comprising:
a heavy chain complementarity determining region 1 (HCDR1) having the amino acid sequence of SEQ ID NO: 1;
HCDR2 having the amino acid sequence of SEQ ID NO: 2; and
HCDR3 having the amino acid sequence of SEQ ID NO: 3; and
a light chain variable domain (VL) having three complementarity-determining regions comprising
a light chain complementarity determining region 1 (LCDR1) having the amino acid sequence of SEQ ID NO: 4;
LCDR2 having the amino acid sequence of SEQ ID NO: 5; and
LCDR3 having the amino acid sequence of SEQ ID NO: 6.

12. The method of claim 5, wherein the ENO-1 monoclonal antibody is used in combination therapy with one or more additional therapeutic agents, comprising: anti-fibrotic agents, immune modulators, or TGF-β pathway inhibitors.

13. The method of claim 5, wherein the TGF-β-driven diseases comprise multiple myeloma, pancreatic cancer, idiopathic pulmonary fibrosis (IPF), bladder cancer, or urothelial carcinoma.
